(19) United States
(12) Patent Application Publication
(10) Pub. No.: US 2021/0361820 A1
(43) Pub. Date: Nov. 25, 2021

(54) ANTIMICROBIAL COMPOSITION, DRESSING, DRESSING COMPONENTS, AND METHOD

(71) Applicants: SYSTAGENIX WOUND MANAGEMENT, LIMITED, West Sussex (GB); KCI USA, INC., San Antonio, TX (US)

(72) Inventors: Katherine A. BOURDILLON, Leeds (GB); Craig DELURY, Gargrave (GB); Sophie REGAN, Skipton (GB); James Sebastian MELLOR, West Sussex (GB); Matthew WESTMORELAND, West Sussex (GB)

(21) Appl. No.: 16/965,721

(22) PCT Filed: Dec. 20, 2018
(86) PCT No.: PCT/US2018/066863

§ 371 Date: Jul. 29, 2020

Related U.S. Application Data
(60) Provisional application No. 62/624,572, filed on Jan. 31, 2018.

(51) Int. Cl. A61L 15/32; A61L 15/28; A61L 15/46; A61L 15/48; C08L 89/06; C08L 1/02; A61L 15/42

(52) U.S. Cl. CPC A61L 15/325; A61L 15/28; A61L 15/46; A61L 2300/404; C08L 89/06; C08L 1/02; A61L 15/425; A61L 15/48

(57) ABSTRACT

An antimicrobial composition may comprise a surfactant. The antimicrobial composition may include about 30 μg/ml to about 1,000 μg/ml of the surfactant, by volume of the antimicrobial composition. The surfactant may comprise a docusate salt such as docusate sodium. The antimicrobial composition may comprise an antimicrobial agent. The antimicrobial composition may include from about 0.01% to about 10% of the antimicrobial agent, by weight of the antimicrobial composition. The antimicrobial agent may comprise polyhexanide (PHMB). In some embodiments, the antimicrobial composition may comprise a matrix-forming material. The antimicrobial composition may include at least 90% of the matrix-forming material, by weight of the antimicrobial composition. The matrix-forming material may comprise collagen, oxidized regenerated cellulose, alginate, carboxymethylcellulose, or combinations thereof.

### CROSS-REFERENCE TO RELATED APPLICATIONS

This application claims the benefit of and priority to U.S. Provisional Patent Application No. 62/624,572, filed Jan. 31, 2018, the entire contents of which are incorporated herein by reference.

### TECHNICAL FIELD

The claimed subject matter relates generally therapy of a tissue site and, more particularly, but without limitation, to compositions and devices, including dressings and dressing components, for application to a tissue site such as a wound, and to methods related to the same. Kits for use in practicing the methods are also provided.

### BACKGROUND

A wide variety of materials and devices, generally characterized as “dressings,” are generally known in the art for use in treating an injury or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage.

Some dressings may protect tissue from, or even assist in the treatment of, infections associated with wounds. Infections can retard wound healing and, if untreated, can result in tissue loss, systemic infections, septic shock, and death. While the benefits of dressings are widely accepted, improvements to dressings may benefit healthcare providers and patients.

### BRIEF SUMMARY

Compositions, for example, in the form of a dressing and methods for using the same, for example, therapy process, are set forth in the appended claims. Illustrative embodiments are also provided to enable a person skilled in the art to make and use the claimed subject matter.

For example, in some embodiments an antimicrobial composition may comprise a surfactant. For example, the antimicrobial composition may include about 30 μg/ml to about 1,000 μg/ml of the surfactant, by volume of the antimicrobial composition. The surfactant may comprise a docusate salt, for example, docusate sodium. In some embodiments, the antimicrobial composition may comprise an antimicrobial agent. For example, the antimicrobial composition may include from about 0.01% to about 10% of the antimicrobial agent, by weight of the antimicrobial composition. The antimicrobial agent may comprise polyhexanide (PHMB). In some embodiments, the antimicrobial composition may comprise a matrix-forming material. For example, the antimicrobial composition may include at least 90% of the matrix-forming material, by weight of the antimicrobial composition. In various embodiments, the matrix-forming material may comprise collagen, oxidized regenerated cellulose, alginate, carboxymethylcellulose, or combinations thereof.

Also, in some embodiments, a dressing may comprise a surfactant. For example, the dressing may include about 30 μg/ml to about 1,000 μg/ml of the surfactant, by volume of the dressing. The surfactant may comprise a docusate salt, for example, docusate sodium. In some embodiments, the dressing may comprise an antimicrobial agent. For example, the dressing may include from about 0.01% to about 10% of an antimicrobial agent, by weight of the dressing. The antimicrobial agent may comprise polyhexanide (PHMB). In some embodiments, the antimicrobial composition may comprise a matrix-forming material. In some embodiments, the dressing may be formed from an antimicrobial composition comprising the surfactant, the antimicrobial agent, and a matrix-forming material. For example, the dressing may in the form of a sponge or a film. For example, the dressing may comprise at least 90% of the matrix-forming material, by weight of the dressing. In various embodiments, the matrix-forming material may comprise collagen, oxidized regenerated cellulose, alginate, carboxymethylcellulose, or combinations thereof. In some embodiments, the dressing may comprise a substrate having an antimicrobial coating. The antimicrobial coating may comprise the surfactant and the antimicrobial agent. In various embodiments, the substrate may comprise a film, a gauze, a mesh, a foam, or combinations thereof.

Also, in some embodiments a method for providing therapy to a tissue site may comprise providing an antimicrobial composition to the tissue site. The antimicrobial composition may comprise a surfactant. For example, the antimicrobial composition may include about 30 μg/ml to about 1,000 μg/ml of the surfactant, by volume of the antimicrobial composition. The surfactant may comprise a docusate salt, for example, docusate sodium. In some embodiments, the antimicrobial composition may comprise an antimicrobial agent. For example, the antimicrobial composition may include from about 0.01% to about 10% of the antimicrobial agent, by weight of the antimicrobial composition. The antimicrobial agent may comprise polyhexanide (PHMB). The antimicrobial composition may form a dressing. For example, the dressing may in the form of a sponge or a film. For example, the dressing may comprise at least 90% of the matrix-forming material, by weight of the dressing. In various embodiments, the matrix-forming material may comprise collagen, oxidized regenerated cellulose, alginate, carboxymethylcellulose, or combinations thereof. In some embodiments, the dressing may comprise a substrate having an antimicrobial coating. The antimicrobial coating may comprise the surfactant and the antimicrobial agent. In various embodiments, the substrate may comprise a film, a gauze, a mesh, a foam, or combinations thereof. In some embodiments, the composition may form an instillation solution or an irrigation solution.

Also provided herein are kits comprising the dressing of any embodiment described herein and instructions for use.

Objectives, advantages, and a preferred mode of making and using the claimed subject matter may be understood best by reference to the accompanying drawings in conjunction with the following detailed description of illustrative embodiments.

### DRAWINGS

FIG. 1 is a cross-sectional, perspective view of an example embodiment a dressing in accordance with this specification.

FIG. 2 is a simplified schematic diagram of an example embodiment of a negative pressure therapy system including the dressing of FIG. 1.

FIG. 3 is a diagrammatic illustration of the reduction in P. aeruginosa after exposure to a collagen/ORC and DSS dressing.

FIG. 4 is a diagrammatic illustration of cell proliferation after exposure to a collagen/ORC and DSS dressing.

FIG. 5 is a diagrammatic illustration of the reduction in P. aeruginosa after exposure to a collagen/ORC, DSS, and PHMB dressing.

It should be noted that the figures set forth herein are intended to illustrate the general characteristics of certain example embodiments. The figures may not precisely reflect the characteristics of any given embodiment, and are not necessarily intended to define or limit the scope of the claimed subject matter.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

Disclosed herein are embodiments of an antimicrobial composition which may be useful in the therapy of a tissue site. Also disclosed herein are embodiments of various components which may be formed from or comprise the antimicrobial composition. For example, also disclosed herein are embodiments of a dressing and dressing components for application to a tissue site and which may comprise the antimicrobial composition. Also disclosed are embodiments of an instillation fluid for application to a tissue site and which may comprise the antimicrobial composition. Also disclosed herein are embodiments of methods related to the disclosed compositions, for example, in the therapy of a tissue site in accordance with disclosure of this specification.

As used herein, “tissue site” is intended to broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue.

The antimicrobial compositions, dressings, and various solutions, and the methods related to the same described herein may provide significant advantages, for example, when employed in the context of a wound therapy regime. Surfactants employed in the dressings, for example, DSS, unexpectedly yields antimicrobial activity. For example, and unexpectedly, the antimicrobial compositions may be advantageously employed to convey antimicrobial properties to various dressings, irrigation solutions, or instillation solutions.

Additionally, while the antimicrobial compositions may convey antimicrobial properties, the antimicrobial compositions have also been found to have no significant negative impact on cellular proliferation at a tissue site. For example, the antimicrobial compositions exhibit no significant cytotoxicity.

### Antimicrobial Composition

In some embodiments, the antimicrobial composition may comprise a surfactant, including a surfactant exhibiting antimicrobial activity. An example of a suitable surfactant is docusate, also known as dioctyl sulfosuccinate (DSS). As used herein, “DSS” is intended to encompass any suitable form. For example, DSS may refer to a docusate salt, for example, docusate sodium, docusate calcium, docusate potassium, or combinations thereof. For example, in a particular embodiment, the surfactant may comprise docusate sodium (C₂₀H₃₇NaO₇S), as shown in the following formula.

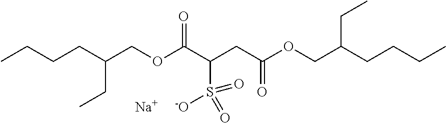

In some embodiments, the surfactant may be present in the antimicrobial dressing composition at a level from about 30 micrograms (μg)/ml to about 1,000 μg/ml, or from about 65 μg/ml to about 250 μg/ml, or about 125 μg/ml of surfactant, by volume of the antimicrobial dressing composition.

In some embodiments, the antimicrobial composition may further comprise various additional, optional components, for example, active materials such as antimicrobial agents, depending upon the intended use or implementation for the antimicrobial composition.

For example, in some embodiments, the antimicrobial composition may also comprise one or more active materials, for example, antimicrobial agents which may be effective to aid in wound healing. Examples of such actives may include non-steroidal anti-inflammatory drugs such as acetaminophen; steroids; antimicrobial agents in addition to the surfactant such as penicillins or streptomycins; antiseptics such as chlorhexidine; and growth factors such as fibroblast growth factor or platelet derived growth factor. If present, such actives may be present at a level from about 0.1% to about 10%, or from about 1% to about 5% by weight of the antimicrobial composition.

In some embodiments, the antimicrobial agents may comprise a safe and effective amount of poly(hexamethylene biguanide) (PHMB), which is also known as polyaminopropyl biguanid (“PAPB”) and polyhexanide, having the following general formula.

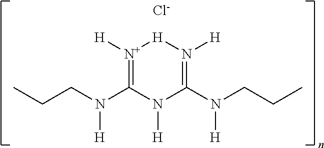

PHMB is a cationic broad spectrum antimicrobial agent. PHMB may be synthesized by a variety of methods, including polycondensation of sodium dicyanamide and hexamethylenediamine.

In some embodiments, the PHMB may be present in the antimicrobial composition at a level from about 0.005% to about 0.025%. In some embodiments, the antimicrobial dressing composition may comprise from about 0.007% to about 0.2%, or more particularly, from about 0.008% to about 0.012%, by weight of the antimicrobial dressing composition. In some embodiments, the antimicrobial dressing composition may comprise the PHMB at a level of about 0.01% by weight of the antimicrobial dressing composition. It has been found that such concentrations of PHMB in combination with the surfactant disclosed herein, may provide previously unrecognized synergistic antimicrobial efficacy while not adversely affecting cell viability or proliferation in wound tissue.

Substrate with Antimicrobial Composition

In some embodiments, the antimicrobial composition may form a dressing or a component thereof. For example, in some embodiments a dressing may be formed from the antimicrobial composition, which may be referred to as an “antimicrobial dressing composition.” In some embodiments, the antimicrobial dressing composition may further comprise one or more additional components or materials. For example, in some embodiments, the antimicrobial dressing composition may comprise a matrix-forming material, such as a material or component that is generally configured to impart structure or form to a dressing formed from the antimicrobial dressing composition.

In some embodiments, the matrix-forming material may comprise a polymeric material, such as a biopolymer or a bioresorbable polymer. As used herein, “bioresorbable” and “bioresorbability” may refer to a characteristic of a material to disintegrate, degrade, or dissolve upon exposure to physiological fluids or processes such that at least a portion of the material may be absorbed. The bioresorbability may be exhibited as a result of a chemical process or condition, a physical process or condition, or combinations thereof. For example, the bioresorbable characteristics of the antimicrobial dressing composition may be such that at least a portion of the dressing may be disintegrated, degraded, or dissolved when in contact with an aqueous medium, such as water, blood or wound exudate. For instance, in various embodiments the antimicrobial layer may be configured such that about 90% by weight, more particularly, about 95% by weight, more particularly, about 99% by weight, more particularly, about 100% by weight of the antimicrobial layer may be disintegrated, degraded, and/or dissolved within a time period from about 24 hours to about 7 days, from introduction into a physiological environment or when incubated with simulated physiological fluid at a temperature of about 37° C.

### Structural Protein (Collagen)

In some embodiments the matrix-forming material may be a structural protein. Examples of suitable structural proteins may include, but are not limited to, fibronectin, fibrin, laminin, elastin, collagen, gelatins, and mixtures thereof. In some embodiments, the matrix-forming material may comprise or may be collagen.

In various embodiments, the collagen may be obtained from any natural source. The collagen may be Type I, II, or III collagen, or may also be chemically-modified collagen, for example an atelocollagen obtained by removing the immunogenic telopeptides from natural collagen. The collagen may also comprise solubilized collagen or soluble collagen fragments, for example, having a molecular weight in the range from about 5,000 to about 100,000, or from about 5,000 to about 50,000, for example, which may be obtained by pepsin treatment of a natural collagen. In various embodiments, the collagen may be obtained from bovine corium that has been rendered largely free of non-collagenous components, for example, including fat, non-collagenous proteins, polysaccharides, and other carbohydrates, as described in U.S. Pat. No. 4,614,794, Easton et al., issued Sep. 30, 1986 and U.S. Pat. No. 4,320,201, Berg et al., issued Mar. 16, 1982, incorporated by reference herein.

In some embodiments, the collagen or other structural protein may be present in the antimicrobial dressing composition at a level from about 1% to about 90% collagen, by weight. In some embodiments, the antimicrobial dressing composition may comprise from about 20% to about 70%, or from about 40% to about 65%, or from about 50% to about 60% collagen, by weight of the antimicrobial dressing composition.

### Polysaccharides, Including Cellulosic Materials (CMC and Alginate)

Additionally or alternatively, in some embodiments the matrix-forming material may be a polysaccharide. Examples of suitable polysaccharides may include hydrocolloids, such as alginic acids and alginic acid salts, guar gum, locust bean gum, pectin, gelatin, xanthum gum, karaya gum, and chitosan. For example, the matrix-forming material may comprise an alginic acid salt such as, but not limited to, calcium alginate, sodium alginate, or combinations thereof. In some embodiments, the alginic acid salt may be present in the antimicrobial dressing composition at a level from about 1% to about 90% alginic acid, by weight. In some embodiments, the antimicrobial dressing composition may comprise from about 5% to about 15%, or about 10%, or from about 40% to about 60% of an alginic acid salt, by weight of the antimicrobial dressing composition.

Further examples of suitable polysaccharides may include cellulosic materials, such as carboxymethylcellulose (CMC) and CMC derivatives, such as sodium CMC, methylcellulose, hydroxymethylcellulose, and combinations thereof, and cellulose ethyl sulphonate (CES). For example, in some embodiments the matrix-forming material may comprise CMC. CMC may be derived from cellulose, for example, where carboxymethyl groups are bonded to hydroxyl groups in the glucopyranose monomers that make up the cellulose. In some embodiments, the CMC may be in salt form, for example, comprising a physiologically acceptable cation, such as sodium (i.e., sodium CMC). CMC is commercially available, such as Walocel™ (sold by The Dow Chemical Company), Cekol® (sold by CP Kelco). In some embodiments, the CMC may be present in the antimicrobial dressing composition at a level from about 1% to about 90% collagen, by weight. In some embodiments, the antimicrobial dressing composition may comprise from about 20% to about 70%, or from about 40% to about 65%, or from about 50% to about 60% of CMC, by weight of the antimicrobial dressing composition.

Still further examples of suitable polysaccharides may include hyaluronic acid and hyaluronic acid salts.

### Oxidized Regenerated Cellulose

Additionally or alternatively, in some embodiments, the matrix-forming material may comprise an oxidized cellulose. In some embodiments, the matrix-forming material may comprise oxidized regenerated cellulose (ORC). Oxidized cellulose may be produced by the oxidation of cellulose, for example with dinitrogen tetroxide. Not intending to be bound by theory, this process may convert primary alcohol groups on the saccharide residues to carboxylic acid groups, for example, forming uronic acid residues within the cellulose chain. The oxidation may not proceed with complete selectivity, and as a result hydroxyl groups on carbons 2 and 3 may be converted to the keto form. These ketone units may introduce an alkali labile link, which at pH 7 or higher initiates the decomposition of the polymer via formation of a lactone and sugar ring cleavage. As a result, oxidized cellulose is biodegradable and bioabsorbable under physiological conditions.

In some embodiments, the oxidized cellulose may be ORC, for example, prepared by oxidation of a regenerated cellulose, such as rayon. The ORC may be manufactured by the process described in U.S. Pat. No. 3,122,479, Smith, issued Feb. 24, 1964, incorporated herein by reference in its entirety. ORC is available with varying degrees of oxidation and hence rates of degradation. In some embodiments, the ORC may be in the form of water-soluble, low molecular weight fragments, for example, obtained by alkali hydrolysis of ORC.

In various embodiments, the ORC may be used in a variety of physical forms, including particles, fibers, a sheet, sponge, or fabrics. In some embodiments, the ORC may be in the form of particles, such as fiber particles or powder particles, for example dispersed in a suitable solid or semisolid topical medicament vehicle. In some embodiments, the ORC may comprise ORC fibers. In some, more particular embodiments, the ORC fibers may have a volume fraction such that at least 80% of the fibers have lengths in the range from about 5 μm to about 1000 μm, or in some more particular embodiments, from about 250 μm to about 450 μm. In various embodiments, a desired size distribution can be achieved, for example, by milling an ORC cloth, followed by sieving the milled powder to remove fibers outside the range. Such fabrics may include woven, non-woven and knitted fabrics.

In some embodiments, the ORC may be present in the antimicrobial dressing composition at a level from about 10% to about 98% of the antimicrobial dressing composition. In some embodiments, the antimicrobial dressing composition may comprise 10% to about 80% ORC, or from about 30% to about 60% ORC, or from about 40% to about 50% ORC, by weigh of the antimicrobial dressing composition.

### Optional Dressing Components

In some embodiments, the antimicrobial dressing composition may optionally further comprise one or more additional materials. Such optional components may include, for example, preservatives, stabilizing agents, plasticizers, matrix strengthening materials, dyestuffs, and combinations thereof.

In some embodiments, the antimicrobial dressing composition may comprise CMC as a modifier, for example, in embodiments where CMC is not already present, which may modify one or more characteristics of the antimicrobial dressing composition, for example, the rheological, absorbency, and other structural characteristics of the antimicrobial dressing composition. CMC may be present in the composition at any level appropriate to result in the desired absorbency and rheological characteristics of the dressing composition.

In some embodiments, the dressing compositions contain a strengthening material, which improves the handling characteristics of the dressing composition by, for example, decreasing its susceptibility to tearing. In a particular embodiment, the strengthening material may comprise non-gelling cellulose fibers. Such “non-gelling” cellulose fibers may be substantially water insoluble and may be produced from cellulose that has not been chemically modified to increase water solubility (e.g., as contrasted from CMC or other cellulose ethers). Non-gelling cellulose fibers are commercially available, such as Tencel® fibers (sold by Lenzing AG). Such fibers may be processed from a commercially-available continuous length, by cutting into lengths that are, in some embodiments, from about 0.5 to about 5 cm, or from about 2 to about 3 cm in length. The non-gelling cellulose fibers may be present in the composition at any level appropriate to result in the desired physical characteristics of the antimicrobial dressing composition. In general, the non-gelling cellulose fibers may be present at a level from about 1% to about 25% of the antimicrobial dressing composition, or more particularly, from about 5% to about 20%, or more particularly, from about 10% to about 15% by weight of the antimicrobial dressing composition.

### Specific Compositions

In some embodiments, the antimicrobial dressing composition may comprise collagen, ORC, and DSS. For example, the antimicrobial dressing composition may comprise from about 50% to about 60% collagen and from about 40% to about 50% ORC by weight of the antimicrobial dressing composition, and from about 30 μg/ml to about 1,000 μg/ml DSS, by volume of the antimicrobial dressing composition. Additionally, in some, still more particular embodiments, the antimicrobial dressing composition may comprise collagen, ORC, DSS, and PHMB. For example, the antimicrobial dressing composition may comprise from about 50% to about 60% collagen and from about 40% to about 50% ORC, and from about 0.005% to about 0.025% PHMB, by weight of the antimicrobial dressing composition, and from about 30 μg/ml to about 1,000 μg/ml DSS by volume of the antimicrobial dressing composition.

In other, more particular embodiments, the antimicrobial dressing composition may comprise collagen, an alginic acid salt (e.g., calcium alginate or sodium alginate), and DSS. For example, the antimicrobial dressing composition may comprise from about 68% to about 94% collagen and from 7% to about 13% calcium alginate and/or sodium alginate, by weight of the antimicrobial dressing composition, and from about 30 μg/ml to about 1,000 μg/ml DSS, by volume of the antimicrobial dressing composition. Additionally, in some, still more particular embodiments, the antimicrobial dressing composition may comprise collagen, an alginic acid salt, DSS, and PHMB. For example, the antimicrobial dressing composition may comprise from about 68% to about 94% collagen and from 7% to about 13% calcium alginate and/or sodium alginate, and from about 0.005% to about 0.025% PHMB, by weight of the antimicrobial dressing composition, and from about 30 μg/ml to about 1,000 μg/ml, by volume of the antimicrobial dressing composition.

Dressing Comprising a Substrate Having a Coating Formed from Antimicrobial Composition

In some embodiments, the antimicrobial composition may form a coating for a substrate. For example, in some embodiments a dressing may comprise a substrate having a coating formed from the antimicrobial composition, which may be referred to as an “antimicrobial coating composition.” In some embodiments, the antimicrobial coating composition may further comprise one or more additional components or materials. For example, in some embodiments, the antimicrobial coating composition may comprise a matrix-forming material. For example, the antimicrobial coating composition may comprise one or more of the matrix-forming materials, as disclosed with respect to the antimicrobial dressing composition. Additionally, in some embodiments of the antimicrobial coating composition may comprise one or more optional components disclosed with respect to the antimicrobial dressing composition, for example, preservatives, stabilizing agents, plasticizers, matrix strengthening materials, dyestuffs, and combinations thereof.

In various embodiments, the substrate to which the antimicrobial coating composition is applied may take any suitable form, examples of which may include, but are not limited to, a nonwoven fibrous substrate such as a mesh or mat; a woven or knitted fibrous substrate such as a gauze; a film; a foam; or combinations thereof.

In some embodiments where the substrate is fibrous, the fibers may be characterized as having an average length from about 0.25 to about 6 inches, or from about 0.5 to about 4 inches, or from about 0.75 to about 3 inches. Also, in some embodiments, the fibers may be characterized as having a denier from about 0.5 to about 40 denier/filament (DPF), or from about 0.75 to about 30 DFP, or from about 1 to about 10 DPF. The woven or nonwoven fibrous substrate may have a suitable thickness, for example, the woven or nonwoven fibrous substrate may have a thickness from about 1 mm to about 25 mm, or in a more particular embodiment, from about 1.5 mm to about 15 mm, more particularly, from about 2 mm to about 12 mm. In various embodiments, the fibers may be characterized as exhibiting antimicrobial activity, as exhibiting absorbency, or combinations thereof. For example, at least a portion of the fibers may comprise antimicrobial fibers, absorbent fibers, of combinations thereof.

Also, in some embodiments, the substrate may comprise a film. In various embodiments, the film may have a thickness within a range from about 400 microns (μm) to about 2,000 μm or, more specifically, a thickness within a range from about 450 μm and about 1,500 μm. For example, the substrate may comprise or be characterized as a film or a thin film. Also, in some embodiments, a substrate comprising a film may be characterized as porous and/or perforated, for example, such that the film may comprise a plurality of pores extending there-through so as to allow fluid communication through the film. For example, the plurality of pores may have an average pore size in the range from about 200 μm to about 3000 μm. Also in some embodiments, the plurality of pores may be present in the film at a pore density in the range from about 2 pores/cm² to about 10 pores/cm².

Also, in some embodiments, the substrate may comprise a foam, for example, a porous foam having interconnected cells or pores that act as flow channels. In various embodiments, the foam may be cellular foam, such as an open-cell foam, which may generally include pores, edges, and/or walls adapted to form interconnected fluid pathways (e.g., channels). The number of pores and the average pore size of the foam may vary according to needs of a prescribed therapy. For example, in various embodiments, the foam may have a porosity from about 20 pores per inch to about 120 pores per inch. Also, in some embodiments, the foam may have an average pore size in a range from about 400 to about 600 microns.

The substrate may be formed from or may comprise any suitable, medically-acceptable material. For example, in some embodiments, the substrate may be characterized as porous and, for example, the substrate may be formed from a porous material such that the substrate may be permeable to a fluid. In some embodiments, the substrate may be characterized as absorbent and, for example, the substrate may be formed from an absorbent material such that the substrate may exhibit absorbency with respect to a fluid, such as water, blood, or wound exudate. In some embodiments, the material may be formed from a bioresorbable material such that the substrate may disintegrate, degrade, or dissolve upon exposure to physiological fluids or processes. Examples of suitable, medically-acceptable materials may include both synthetic materials and natural materials such as proteins and biopolymers.

For example, in some embodiments the substrate may comprise a synthetic material, examples of which include acrylates and acrylic resins, polyurethanes, silicone and silicone resins, polyamides, a polyolefins, polyesters, polypropylenes, and polyethylenes, such as high-molecular-weight polyethylene (HMWP). In some embodiments, the base fibers may be formed from a polyamide, such as nylon. In some embodiments, the substrate may be a hydrophobic material, for example, a reticulated polyurethane foam such as the foam employed in the V.A.C.® GRANUFOAM™ Dressing available from Acelity, Inc. of San Antonio, Tex.

Additionally or alternatively, the substrate may comprise a natural material. For example, the natural material may comprise a structural protein such as fibronectin, fibrin, laminin, elastin, collagen, or a gelatin. Additionally or alternatively, the natural material may comprise a polysaccharide, examples of which may include hydrocolloids such as alginic acids and alginic acid salts, guar gum, locust bean gum, pectin, gelatin, xanthum gum, karaya gum, and chitosan, and cellulosic materials such as CMC, CMC derivatives, and ORC.

Also for example, in some embodiments, the substrate may be made from a hydrophilic material capable of wicking fluid away from a tissue site, for example, by drawing fluid away from the tissue site by capillary flow or other wicking mechanisms. An example of a hydrophilic foam is a polyvinyl alcohol, open-cell foam such as the foam employed V.A.C. WHITEFOAM™ Dressing available from Acelity, Inc. of San Antonio, Tex. Other hydrophilic foams may include those made from a polyether. Other foams that may exhibit hydrophilic characteristics include hydrophobic foams that have been treated or coated to provide hydrophilicity. Other examples of absorbent, polyurethane dressings suitable for use as the substrate disclosed herein may include hydropolymer foam dressings such as the TIELLE™ Silicone Border Hydropolymer Dressing, the TIELLE™ Plus Hydropolymer Adhesive dressing, the TIELLE™ Lite Hydropolymer Adhesive Dressing, the TIELLE™ Packing Hydropolymer Non-Adhesive Dressing, the TIELLE™ Non-Adhesive Hydropolymer Dressing, and the TIELLE™ Border Adhesive Hydropolymer Dressing, all available from Acelity, Inc. of San Antonio, Tex.

Also in some embodiments, the substrate may comprise a freeze-dried matrix comprising collagen and ORC. An example of a freeze-dried collagen and ORC dressing suitable for use as the substrate disclosed herein is the PROMOGRAN™ Matrix Wound Dressing available from Acelity, Inc. of San Antonio, Tex.

In another embodiment, the substrate may comprise calcium alginate and CMC fibers, as example of which is the SILVERCEL™ Antimicrobial Alginate Dressing and the SILVERCEL™ NON-ADHERENT Antimicrobial Alginate Dressing available from Acelity, Inc. of San Antonio, Tex.

In another embodiment, the substrate may comprise sodium CMC and strengthening cellulose fibers in combination, an example of which is the BIOSORB™ Gelling Fiber Dressing available from Acelity, Inc. of San Antonio, Tex. Additional examples of suitable substrates, suitable for use herein, will be appreciated by those of ordinary skill in the art upon viewing this disclosure.

### Dressings

In some embodiments, the dressing may be generally configured to be in contact with the tissue site. For example, the dressing may be configured so as to be in contact with a portion of a tissue site, substantially all of a tissue site, or a tissue site in its entirety. If a tissue site is a wound, for example, the dressing may partially or completely fill a wound, or may be placed over a wound. In various embodiments, the dressing may take many forms, and may have many sizes, shapes, or thicknesses depending on a variety of factors, such as the type of treatment being implemented or the nature and size of a tissue site. For example, the size and shape of the dressing may be adapted to the contours of deep and irregular shaped tissue sites and/or may be configured so as to be adaptable to a given shape or contour. Moreover, in some embodiments, any or all of the surfaces of the dressing may comprise projections or an uneven, course, or jagged profile that can, for example, induce strains and stresses on a tissue site, for example, which may be effective to promote granulation at the tissue site.

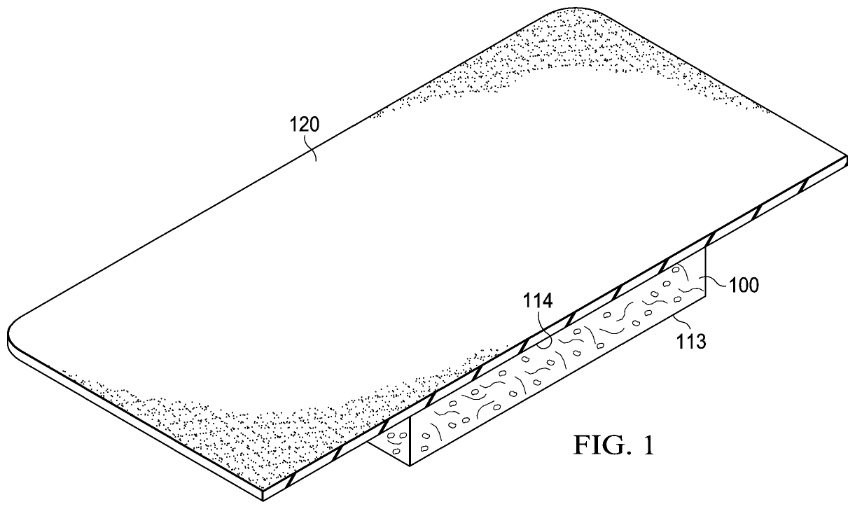

In some embodiments, the dressing may be in substantially sheet form. For example, in the embodiment of FIG. 1, a dressing 100 may comprise a generally planar structure having two opposite-facing planar surfaces and a depth or thickness orthogonal to the planar surfaces. For example, the dressing 100 may comprise a first surface 113 and a second surface 114. The first surface 113 may be configured to face a tissue site, and the second surface 114 may be opposite the first surface 113. The first surface 113 and/or second surface 114 may have a surface area from about 1 cm² to about 400 cm², from about 2 cm² to about 200 cm², or from about 4 cm² to about 100 cm². In various embodiments, the first surface 113 and the second surface 114 may have any suitable shape, examples of which include but are not limited to, triangles, squares, rectangles, ellipses, circles, ovals, and various polygons having four, five, six, seven, eight, or more sides. The shape and area of the first surface 113 and the second surface 114 may be customized to the location and type of tissue site onto which the dressing 100 is to be applied.

In some embodiments, the dressing 100 may comprise one or more additional layers. For example, in various embodiments, such additional layers may perform any of a variety of functions including, for example, adherence of the dressing 100 to a tissue site or to surrounding tissues, increasing structural rigidity of the dressing, protection from moisture or other materials in the external environment, protection of a wound surface, delivery of one or more actives or other materials to the wound surface, or combinations thereof. In various embodiments, the additional layers may be conformable to a wound surface and/or to the surrounding tissues, for example, being capable of bending such that the wound-facing surfaces of the dressing are in substantial contact with the wound and/or the surrounding tissues.

For example, in the embodiment of FIG. 1, the dressing 100 further may be used with a cover 120 having a first surface and a second surface. The cover 120 may support the dressing 100 on the first surface of the cover 120, for example, such that the second surface 114 of the dressing 100 is proximate to and in contact with the first surface of the cover 120.

In some embodiments, the cover 120 may generally be configured to provide a bacterial barrier and protection from physical trauma. The cover 120 may be constructed from a material that can reduce evaporative losses and provide a fluid seal between two components or two environments, such as between a therapeutic environment and a local external environment. The cover 120 may be, for example, an elastomeric film or membrane that can provide a seal at a tissue site for a given negative-pressure source. In some embodiments, the cover 120 may have a high moisture-vapor transmission rate (MVTR). For example, in some embodiments, the MVTR may be at least 300 g/m² per twenty-four hours. In some embodiments, the cover 120 may be formed from a suitable polymer. For example, the cover 120 may comprise a polymer drape, such as a polyurethane film, that may be permeable to water vapor but generally impermeable to liquid. In some embodiments, the cover 120 may have a thickness in the range of about from 25 to about 50 microns.

In some embodiments, the cover 120 may be configured to be attached to an attachment surface, such as undamaged epidermis, a gasket, or another cover, for example, via an attachment device. For example, in some embodiments, the cover 120 may be attached to tissue proximate a tissue site, such as epidermis, so as to form a sealed space. In such an embodiment, the attachment device may take any suitable form. For example, an attachment device may be a medically-acceptable, pressure-sensitive adhesive that extends about a periphery, a portion, or an entire surface of the cover 120. In some embodiments, for example, some or all of the cover 120 may be coated with an adhesive, such as an acrylic adhesive, having a coating weight between 25-65 grams per square meter (g.s.m.). Thicker adhesives, or combinations of adhesives, may be applied in some embodiments, for example, to improve the seal and reduce leaks. Other examples of an attachment device may include a double-sided tape, a paste, a hydrocolloid, a hydrogel, a silicone gel, or an organogel.

### Dressing—Secondary Layer

Additionally, in some embodiments, the dressing 100 may further comprise a secondary layer, for example, positioned between the dressing 100 and the cover 120. In some embodiments, the secondary layer may comprise fluid pathways interconnected so as to improve distribution or collection of fluids. For example, in some embodiments, the secondary layer may be a porous foam material having a plurality of interconnected cells or pores, edges, and/or walls, to form interconnected fluid pathways (e.g., channels). Non-limiting examples include cellular foam, open-cell foam, reticulated foam, porous tissue collections, and other porous material such as gauze or felted mat. For example, in some embodiments, the secondary layer may be a foam having pore sizes in a range of 400-600 microns. In one non-limiting example, the secondary layer may be an open-cell, reticulated polyurethane foam.

In some embodiments where the secondary layer is present, the secondary layer may be characterized as exhibiting absorbency. For example, the secondary layer may exhibit an absorbency of at least 3 g saline/g, more particularly, at least 5 g saline/g, more particularly, from 8 to 20 g saline/g. In some embodiments, the secondary layer may be hydrophilic and may also absorb, for example, wick fluid away from a dressing 100. In such an embodiment, the wicking properties of the secondary layer may draw fluid away from the dressing 100 by capillary flow or other wicking mechanisms. An example of a hydrophilic foam is a polyvinyl alcohol, open-cell foam. Other hydrophilic foams may include those made from polyether. Other foams that may exhibit hydrophilic characteristics include hydrophobic foams that have been treated or coated to provide hydrophilicity.

### Negative—Pressure Therapy

Additionally, in some embodiments, a dressing or dressing layer, such as the dressing 100, may be employed in therapy in which a tissue site is treated with reduced pressure. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure.” Negative-pressure therapy may provide a number of benefits, including migration of epithelial and subcutaneous tissues, improved blood flow, and micro-deformation of tissue at a wound site. Together, these benefits may increase development of granulation tissue and reduce healing times.

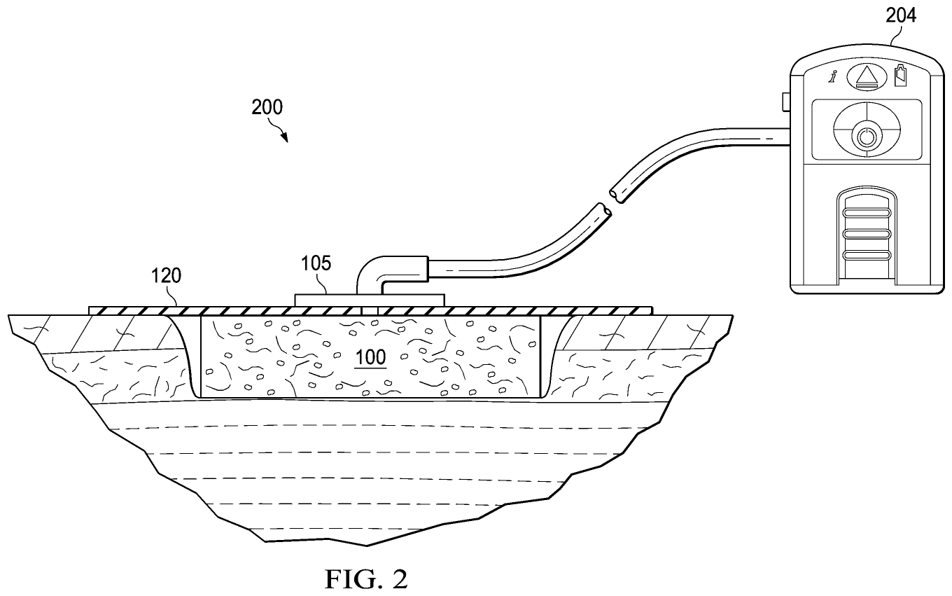

For example, FIG. 2 illustrates an embodiment of a negative-pressure therapy system 200 in a simplified schematic. Generally, the negative-pressure therapy system 200 may be configured to provide negative-pressure to a tissue site. In various embodiments, a negative-pressure therapy system generally includes a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a tissue site. For example, in the embodiment of FIG. 2, the dressing 100 is fluidly coupled to a negative-pressure source 204 such that negative pressure may be applied to a tissue site via the dressing 100.

The dressing 100 may be generally configured to distribute negative pressure, to collect fluid, or both. For example, in some embodiments, the dressing 100 may comprise or be configured as a manifold. A “manifold” in this context generally includes any composition or structure providing a plurality of pathways configured to collect or distribute fluid across a tissue site under pressure. For example, a manifold may be configured to receive negative pressure from the negative-pressure source 204 and to distribute negative pressure through multiple apertures or pores, which may have the effect of collecting fluid and drawing the fluid toward the negative-pressure source 204. More particularly, in the embodiment of FIG. 2, the dressing 100 is configured to receive negative pressure from the negative-pressure source 204 and to distribute the negative pressure through the dressing 100, for example, which may have the effect of collecting fluid from a sealed space by drawing fluid from a tissue site through the dressing 100. In additional or alternative embodiments, the fluid path(s) may be reversed or a secondary fluid path may be provided to facilitate movement of fluid across a tissue site. In some embodiments, the fluid pathways of a manifold may be interconnected to improve distribution or collection of fluids. In some embodiments, a manifold may be a porous foam material having a plurality of interconnected cells or pores. For example, open-cell foam may generally include pores, edges, and/or walls that may form interconnected fluid pathways, such as channels.

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, wound exudate and other fluid flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

As used herein, “negative pressure” is generally intended to refer to a pressure less than a local ambient pressure, such as the ambient pressure in a local environment external to a sealed therapeutic environment provided by the dressing 100. In many cases, the local ambient pressure may also be the atmospheric pressure proximate to or about a tissue site. Alternatively, the pressure may be less than a hydrostatic pressure associated with tissue at a tissue site. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

In various embodiments, a negative-pressure supply, such as the negative-pressure source 204, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 204 may be combined with a controller and other components into a therapy unit. A negative-pressure supply may also have one or more supply ports configured to facilitate coupling and de-coupling of the negative-pressure supply to one or more distribution components.

In various embodiments, components may be fluidly coupled to each other to provide a path for transferring fluids (i.e., liquid and/or gas) between the components. For example, components may be fluidly coupled through a fluid conductor. As used herein, the term “fluid conductor” is intended to broadly include a tube, pipe, hose, conduit, or other structure with one or more lumina adapted to convey a fluid between two ends thereof. Typically, a fluid conductor may be an elongated, cylindrical structure with some flexibility, but the geometry and rigidity may vary. In some embodiments, the negative-pressure source 204 may be operatively coupled to the dressing 100 via a dressing interface. For example, in the embodiment of FIG. 2, the dressing 100 may be coupled to the negative-pressure source 204 via a dressing interface 105 such that the dressing 100 receives negative pressure from the negative pressure source.

### Instillation Fluid

In some embodiments, the antimicrobial composition may form a fluid for application to a tissue site. For example, in some embodiments the antimicrobial composition may form an irrigation solution and/or an instillation solution for application to a wound. In various embodiments, the irrigation and/or instillation solution may comprise an aqueous solution such as a sterile-water solution; a saline-containing solution; a hydrogen peroxide-containing solution; a sodium hypochlorite-containing solution; an alcohol-containing solution; a povidone iodine-containing solution; a silver nitrate-containing, for example, a 0.5% silver nitrate solution; a sulfur-containing solution; a biguanide-containing solution; a lidocaine-containing solution; a lavasept-containing solution; an acetic acid-containing solution; a betaine-containing solution; a bacitracin or nebacetin-containing solution; or combinations thereof.

### Methods of Use

Also, in some embodiments, a method for treating a tissue site with the antimicrobial composition may generally comprise providing the antimicrobial composition comprising the surfactant to a tissue site.

For example, in some embodiments where the antimicrobial composition forms the dressing 100, providing the antimicrobial composition to a tissue site may comprise applying the dressing 100 to a tissue site. For example, the dressing 100 may be placed within, over, on, or otherwise proximate to a tissue site. Additionally, in some embodiments, a cover, such as the cover 120, may be placed over the dressing 100, and the cover can be sealed to an attachment surface near the tissue site. For example, the dressing 100 may be sealed to undamaged epidermis peripheral to a tissue site. In some embodiments, the dressing 100 may be positioned first and, after the dressing 100 has been positioned, the cover 120 may be positioned. In some other embodiments, the dressing 100 and cover 120 may be preassembled, for example, such that the dressing 100 and cover 120 are positioned with respect to each other prior to placement proximate a tissue site. Thus, the dressing 100 and the cover 120 can provide a sealed therapeutic environment proximate to a tissue site, substantially isolated from the external environment.

Additionally or alternatively, in some embodiments where the dressing 100 is employed in the context of a negative-pressure therapy, the negative-pressure therapy may further comprise fluidly coupling a negative-pressure source to the sealed space and operating the negative-pressure source to generate a negative pressure in the sealed space. For example, the negative-pressure source 204 may be coupled to the dressing 100 such that the negative-pressure source 204 may be used to reduce the pressure in the sealed space. For example, negative pressure applied across a tissue site, for example, via the dressing 100 may be effective to induce macrostrain and microstrain at the tissue site, as well as remove exudates and other fluids from the tissue site.

### Instillation/Irrigation Therapy

In embodiments where the antimicrobial composition forms an instillation or irrigation solution, providing the surfactant to a tissue site may comprise communicating the instillation solution or the irrigation solution from a solution source to the tissue site. For example, in various embodiments, an instillation solution may be drawn or pumped from a solution source. For example, a volume of the instillation solution within a container may be provided to a tissue site as a part of a negative-pressure therapy. Additionally or alternatively, an irrigation solution may be drawn or pumped to a wound, for example, to improve wound hydration, remove cellular debris and/or aid in debridement, provide cellular nutrients or antimicrobial agents, or combinations thereof.

### Methods of Making

Methods of preparing the dressings as described herein are also provided. The method may comprise adding a solution comprising the surfactant as described herein (e.g., DSS) to an intermediate slurry comprising a matrix-forming material as described herein to form a biomaterial slurry. The solution comprising the surfactant (e.g., DSS) may be prepared by mixing a suitable amount of the surfactant as described herein (e.g., DSS), for example, in powdered form or liquid form, with a solvent, such as water, to form the solution comprising the surfactant as described herein (e.g., DSS) in a suitable concentration such that the resultant biomaterial, after mixing with the intermediate slurry, has a surfactant (e.g., DSS) concentration as described herein as described herein.

In various embodiments, the methods described herein may further comprise drying or dehydrating the biomaterial slurry, for example, to form a sponge or a film. Drying may comprise freeze-drying or solvent-drying of the biomaterial slurry. Freeze-drying may comprise the steps of freezing the biomaterial slurry, followed by evaporating the solvent from the frozen biomaterial slurry under reduced pressure. Suitably, a method of freeze-drying is similar to that described for a collagen-based sponge in U.S. Pat. No. 2,157,224, the entire content of which is incorporated herein by reference. In some embodiments, the freeze-drying may be performed in stages to prepare the multi-layered configurations described herein. In some embodiments, a first layer comprising biomaterial as described herein may be frozen at a suitable temperature until solid, for example about −80° C. A second layer comprising biomaterial as described herein may be added adjacent to the first layer by repeating the process until a desired composition is achieved. The resultant multi-layered configuration may be freeze-dried as described above.

Solvent-drying may comprise freezing the biomaterial slurry, followed by immersing the biomaterial slurry in a series of baths of a hygroscopic organic solvent such as anhydrous isopropanol to extract the water from the frozen biomaterial slurry, followed by removing the organic solvent by evaporation. Methods of solvent drying are described, for example, in U.S. Pat. No. 3,157,524, the entire content of which is incorporated herein by reference.

In some embodiments, to form a biomaterial film as described herein, the biomaterial slurry as prepared as described above, may be placed in a dehydration oven, which may evaporate water and/or solvent using suitably higher temperatures with or without circulation of air through a chamber containing a desiccant or the like.

In some embodiments, the methods may further comprise treating the biomaterial slurry, or the dried biomaterial, with a cross-linking agent such as epichlorhydrin, carbodiimide, hexamethylene diisocyanate (HMDI) orglutaraldehyde. Alternatively, cross-linking may be carried out dehydrothermally. The method of cross-linking can affect the final product. For example, HMDI cross-links the primary amino groups on collagen, whereas carbodiimide cross-links carbohydrate on the ORC to primary amino groups on the collagen.

Surfactants employed in the dressings, for example, DSS, unexpectedly yields antimicrobial activity. For example, and unexpectedly, the antimicrobial compositions may be advantageously employed to convey antimicrobial properties to various dressings, irrigation solutions, or instillation solutions. Additionally, surfactants such as DSS employed in the dressings, in combination with PHMB unexpectedly yields synergistic antimicrobial activity. For example, the combination of DSS and PHMB unexpectedly demonstrates an improved antimicrobial activity in comparison to the antimicrobial activity of either DSS or PHMB, alone.

Additionally, while the antimicrobial compositions may convey antimicrobial properties, the antimicrobial compositions have also been found to have no significant negative impact on cellular proliferation at a tissue site. For example, the antimicrobial compositions exhibit no significant cytotoxicity.

### Kits

The present disclosure provides kits that include a dressing of any embodiment described herein and instructions for use. The kit may optionally include instructions for generating a dressing of any embodiment described herein. The kits of the present technology may also include methods for treating a wound or a tissue site in a subject in need thereof. The kit may optionally comprise components such as antiseptic wipes, ointment, adhesive tape, tweezers, or scissors.

### EXAMPLES

The advantages associated with the disclosed antimicrobial compositions, dressings, and various solutions are further demonstrated by the following, non-limiting examples. These examples may demonstrate one or more features associated with some embodiments of the dressings, and systems.

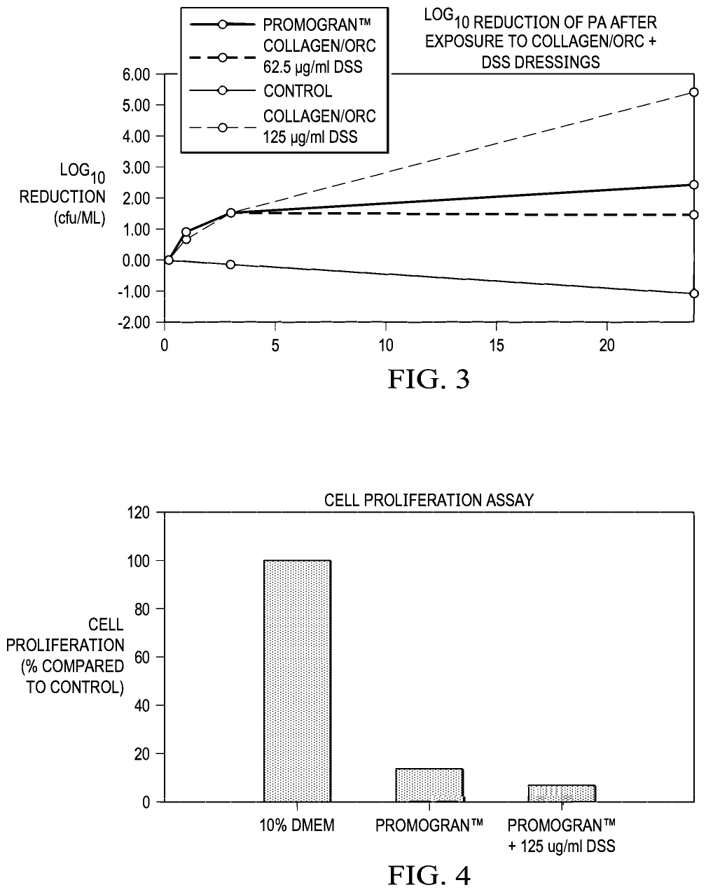

In Example 1, DSS was added to collagen and ORC at varying concentrations to yield collagen/ORC and DSS dressings having a concentration of 62.5 μg of DSS and 125 μg of DSS, respectively, per ml of the dressing. The dressings were evaluated with respect to a commercially to determine the effect of those dressings on microbial activity, for example, P. aeruginosa. FIG. 3 illustrates the Log₁₀ reduction in P. aeruginosa after exposure to the collagen/ORC and DSS dressings, particularly, a Control, a commercially-available collagen/ORC dressing (a PROMOGRAN™ Matrix Wound Dressing), a collagen/ORC and DSS dressing having 62.5 μg of DSS, and a collagen/ORC and DSS dressing having 125 μg of DSS. As shown in FIG. 3, the dressing having 125 μg/ml of DSS exhibited significant antimicrobial activity, yielding greater than 5 Log₁₀ unit reduction in P. aeruginosa within 24 hours. This antimicrobial activity was unexpected.

In Example 2, the dressing comprising 125 μg/ml of DSS was evaluated with respect to cell proliferation. FIG. 4 illustrates the effect on cell proliferation after exposure of the collagen/ORC and DSS dressing having 125 μg of DSS in comparison to a PROMOGRAN™ Matrix Wound Dressing, and a 10% Dulbecco's Modified Eagle Medium (DMEM). As shown in FIG. 4, the dressing having 125 μg/ml of DSS allowed cell proliferation and exhibited no significant difference in cellular proliferation with respect to the PROMOGRAN™ Matrix Wound Dressing. These results indicate that the dressing having 125 μg DSS/ml of collagen and ORC does not exhibit any significant cytotoxicity.

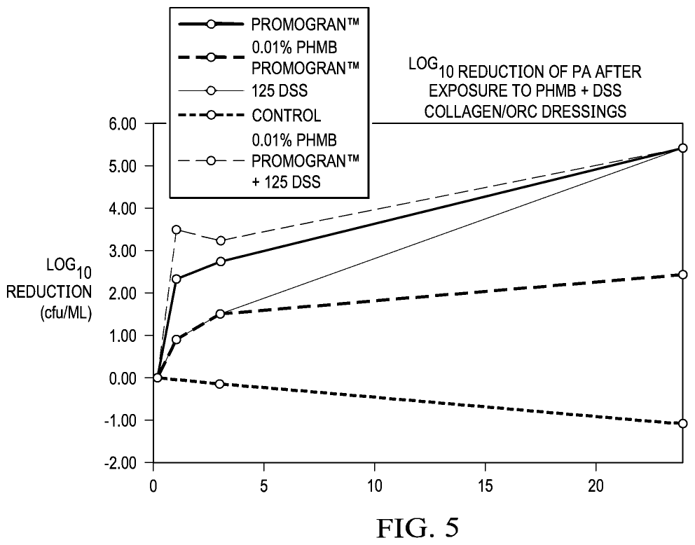

In Example 3, the collagen/ORC and DSS dressing comprising 125 μg/ml of DSS was compared to a collagen/ORC dressing comprising 0.01% PHMB. Also, a collagen/ORC dressing comprising 125 μg/ml of DSS and 0.01% PHMB was also prepared and evaluated. FIG. 5 illustrates the Log₁₀ reduction in P. aeruginosa after exposure to these dressings. As shown in FIG. 6, the collagen/ORC and DSS dressing comprising 125 μg/ml of DSS and 0.01% PHMB exhibited improved antimicrobial activity in comparison to the dressings including DSS alone or PHMB alone, indicating a synergistic interaction where both DSS and PHMB are present within the collagen/ORC dressing. Additionally, FIG. 5 indicates that the synergistic combination of collagen/ORC DSS, and PHMB, may be employed to further improve antimicrobial activity of a dressing.

### Non-Limiting Discussion of Terminology

The description and specific examples above, while providing illustrative embodiments, are intended for purposes of illustration only and are not intended to limit the scope of the claimed subject matter. Moreover, recitation of multiple embodiments having stated features is not intended to exclude other embodiments having additional features, or other embodiments incorporating different combinations of the stated features. Components may be also be combined or eliminated in various configurations for purposes of sale, manufacture, assembly, or use. Specific examples are provided for illustrative purposes of how to make and use the compositions and methods of claimed subject matter and, unless explicitly stated otherwise, are not intended to be a representation that given embodiments of claimed subject matter have, or have not, been made or tested. Equivalent changes, modifications and variations of some embodiments, materials, compositions and methods can be made within the scope of the appended claims, with substantially similar results.

As used herein, the word “include,” and its variants, is intended to be non-limiting, such that recitation of items in a list is not to the exclusion of other like items that may also be useful in the materials, compositions, devices, and methods of the claimed subject matter. Similarly, the terms “can” and “may” and their variants are intended to be non-limiting, such that recitation that an embodiment can or may comprise certain elements or features does not exclude other embodiments of the claimed subject matter that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe and claim certain embodiments, embodiments may alternatively be described using more limiting terms such as “consisting of” or “consisting essentially of.” Thus, for any given embodiment reciting materials, components or process steps, the claimed subject matter may also specifically include embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of this disclosure. For example, the term “about” may refer to deviations that are greater or lesser than a stated value or range by 1/10 of the stated value(s), e.g., ±10%, as appropriate from the context of the disclosure. For instance, a concentration value of “about 30%” may refer to a concentration between 27% and 33%. Whether or not modified by the term “about,” quantitative values recited in the claims include equivalents to the recited values, for example, deviations from the numerical quantity, as would be recognized as equivalent by a person skilled in the art in view of this disclosure.

The appended claims set forth novel and inventive aspects of the subject matter disclosed and described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. An antimicrobial composition comprising:
from about 30 μg/ml to about 1,000 μg/ml of a surfactant, by volume of the composition;
from about 0.01% to about 10% of an antimicrobial agent, by weight of the antimicrobial composition; and
at least 90% of a matrix-forming material, by weight of the antimicrobial composition.

2. The antimicrobial composition of claim 1, wherein the surfactant comprises a docusate salt.

3. The antimicrobial composition of claim 2, wherein the docusate salt comprises docusate sodium.

4. The antimicrobial composition of claim 1, wherein the matrix-forming material is bioresorbable.

5. The antimicrobial composition of claim 1, wherein the matrix-forming material comprises collagen.

6. The antimicrobial composition of claim 1, wherein the matrix-forming material comprises from about 50% to about 60% collagen and about 40% to about 50% oxidized, regenerated cellulose (ORC) by weight of the antimicrobial composition.

7. The antimicrobial composition of claim 1, wherein the matrix-forming material comprises 90% collagen and 10% calcium alginate by weight of the antimicrobial composition.

8. The antimicrobial composition of claim 1, wherein the matrix-forming material comprises cellulose.

9. The antimicrobial composition of claim 1, wherein the matrix-forming material comprises carboxymethylcellulose.

10. The antimicrobial composition of claim 1, wherein the antimicrobial agent comprises polyhexanide (PHMB).

11. The antimicrobial composition of claim 10, wherein the antimicrobial composition comprises from about 0.005% to about 0.025% PHMB, by weight of the antimicrobial composition.

12. A dressing comprising
the antimicrobial composition of claim 1.

13-21. (canceled)

22. The dressing of claim 12, wherein the dressing comprises a sponge or a film.

23. The dressing of claim 12, wherein the dressing comprises a substrate having the antimicrobial coating.

24. The dressing of claim 23, wherein the substrate comprises a film.

25. The dressing of claim 23, wherein the substrate comprises gauze.

26. The dressing of claim 23, wherein the substrate comprises a mesh.

27. The dressing of claim 23, wherein the substrate comprises a foam.

28-29. (canceled)

30. A method for providing therapy to a tissue site, the method comprising providing an antimicrobial composition to the tissue site, the antimicrobial composition comprising:
from about 30 μg/ml to about 1,000 μg/ml of a surfactant, by volume of the composition; and
from about 0.01% to about 10% of an antimicrobial agent, by weight of the antimicrobial composition.

31-52. (canceled)

53. A system for treating a tissue site with reduced pressure, the system comprising
a dressing comprising:
from about 30 μg/ml to about 1,000 μg/ml of a surfactant, by volume of the dressing; and
from about 0.01% to about 10% of an antimicrobial agent, by weight of the dressing; and
a reduced-pressure source adapted to be fluidly coupled to the dressing.

54-71. (canceled)
